(19) United States
(12) Patent Application Publication
(10) Pub. No.: US 2009/0246258 A1
(43) Pub. Date: Oct. 1, 2009

(54) Antimicrobial and odor adsorbing textile

(76) Inventors: Piyush Shukla, Greer, SC (US); Michael Anderson, Spartanburg, SC (US)

Correspondence Address: Legal Department (M-495), P.O. Box 1926, Spartanburg, SC, 29304, US

(21) Appl. No.: 12/079,809

(22) Filed: Mar. 28, 2008

(51) Int. Cl. A61K 9/70; A61P 31/00

(52) U.S. Cl. 424/443

(57) ABSTRACT

The antimicrobial and odor adsorbing fabric substrate has a surface and at least a portion of the surface is coated with a finish. The finish contains a compound selected from the group consisting of silver particle-containing compounds, silver ion-containing compounds, silver ion-generating compounds, and any combinations thereof, a hyperbranched polyethyleneimine derivative, potassium citrate, inorganic chloride, a polyurethane binder, and a cross-linking agent. The silver-ion containing compound is selected from the group consisting of silver zirconium phosphate, silver zeolite, silver glass, and any mixtures thereof or a conductive silver containing nanoparticle. The hyperbranched polyethyleneimine derivative is of the formula:

(R)ₓ−h-PEI−(A)_{y}
where R is a non-hyperbranched hydrocarbon group and the hydrocarbon group has at least one linear portion. The linear portion has between 5 and 30 carbon atoms, x is a number from 1 to 10,000, h-PEI is a hyperbranched polyethyleneimine, A is an organic compound having from 1 to 4 carbon atoms, and y is a number from 0 to 500.

### FIELD OF THE INVENTION

The present application is directed towards antimicrobial and odor adsorbing textiles.

### BACKGROUND

It has long been desirable to produce a textile substrate having durable odor adsorption capabilities. In particular, the abatement of human sweat odors is useful in a variety of different applications. For instance, hunters are interested in preventing their body odors from reaching animals being pursued. In perhaps a more common application, apparel that may be worn several times before requiring laundering would provide considerable benefits to users thereof.

It has also been desirable to deter odors by the treatment of the textile article with antimicrobial compounds or the incorporation of antimicrobial compounds into the yarns used to make the substrate. Antimicrobial-treated textiles function to reduce odors by controlling or preventing the growth of microorganisms. When microorganisms grow, they degrade materials into volatile organic compounds, which are often malodorous.

One approach used to prevent odors has been to incorporate carbon black particles, granules, fibers, or cloths into a textile. Depending on the chosen pore size and source material, carbon black is generally effective at adsorbing odors when dry, but it tends to lose some of its efficacy when wet (as the surface area becomes blocked by water or other aqueous contaminants). A second problem with carbon black is that it must be bound to the textile by adhesives or other binders, often reducing the breathability of the treated textile. Finally, carbon black imparts a black color to the textile surface being treated, which is unsuitable in many situations (for example, with light-colored apparel).

Thus there is a need for a textile having both antimicrobial and odor adsorbing characteristics that is also wash durable.

### SUMMARY OF THE INVENTION

The present invention provides an antimicrobial and odor adsorbing fabric substrate that has a surface and at least a portion of the surface is coated with a finish. The finish contains a compound selected from the group consisting of silver particle-containing compounds, silver ion-containing compounds, silver ion-generating compounds, and any combinations thereof, a hyperbranched polyethyleneimine derivative, potassium citrate, inorganic chloride, a polyurethane binder, and a cross-linking agent. The hyperbranched polyethyleneimine derivative is of the formula:

(R)ₓ−h-PEI−(A)_{y}

where R is a non-hyperbranched hydrocarbon group and the hydrocarbon group has at least one linear portion. The linear portion has between 5 and 30 carbon atoms, x is a number from 1 to 10,000, h-PEI is a hyperbranched polyethyleneimine, A is an organic compound having from 1 to 4 carbon atoms, and y is a number from 0 to 500.

### DETAILED DESCRIPTION OF THE INVENTION

The finish applied to the fabric substrate imparts odor control properties to the treated textiles by controlling microbes and adsorbing odors (specifically short chain fatty acids, a major component of body odor). Additionally, the chemical treatment provides benefits in terms of reduced drying time, reduced wrinkling, and, in some circumstances, improved moisture wicking.

Any fabric (yarns or textiles) may be utilized as the substrate within this application. Thus, natural (cotton and the like) or synthetic fibers (polyesters, polyamides, polyolefins, and the like) may constitute the target substrate, either by itself or in any combinations or mixtures of synthetics, naturals, or blends or both types. As for the synthetic types, for instance, and without intending any limitations therein, polyolefins, such as polyethylene, polypropylene, and polybutylene, halogenated polymers, such as polyvinyl chloride, polyesters, such as polyethylene terephthalate, polyester/polyethers, polyamides, such as nylon 6 and nylon 6,6, polyurethanes, polyaramids, such as KEVLAR® and NOMEX® from DuPont, as well as homopolymers, copolymers, or terepolymers in any combination of such monomers, and the like, may be utilized within this invention. Nylon 6, Nylon 6,6, polyaramids, polypropylene, and polyethylene terephthalate (a polyester) are particularly preferred with the cross-linked binder systems of this invention, particularly due to the surface modifications provided by such cross-linked systems. The fabrics may be knit, woven, or nonwoven.

The antimicrobial and odor adsorbing fabric substrate has a surface, at least a portion of which is coated with a finish. The finish contains a compound selected from the group consisting of silver particle-containing compounds, silver ion-containing compounds, silver ion-generating compounds, and any combinations thereof, a hyperbranched polyethyleneimine derivative, potassium citrate, inorganic chloride, a polyurethane binder, and a cross-linking agent.

The current commercial products using the odor-adsorbing treatment include a softener package that is high in sodium, a compound that is deleterious to the antimicrobial finish. Moreover, the current commercial antimicrobial finish includes an acidic softener system that causes the odor-adsorbing treatment to lose its homogeneity. Additionally, one of the binder systems used to achieve wash durability of the antimicrobial finish is not compatible with the PEI or the potassium citrate. The alternative binder system used in the antimicrobial finish, however, is also not compatible as it leads to loss of colorfastness. And without the binder system, wash durability can not be achieved. Due to the sensitivity of the antimicrobial finish to certain metals (namely, sodium) and of the odor control finish to pH, challenges were faced when combining the two. A variety of softeners and binder systems and their respective ratios had to be explored to discover an appropriate system amenable to both treatments. Furthermore, the curing of the odor adsorbing finish at elevated temperatures leads to fading of printed materials. Processing parameters had to be explored that would both durably cure the treatments as well as not lead to fading of the goods.

The treated substrates exhibit durable odor control, even after multiple launderings. Additionally, and surprisingly, the substrates also have durable softness, reduced wrinkling after laundering/drying, and improved moisture wicking capability.

One component of the finish is a modified hyperbranched polyethyleneimine compound, also referred to herein as a “hyperbranched polyethyleneimine derivative.” The hyperbranched polyethyleneimine derivative, or h-PEI, comprises at least one hyperbranched polyethyleneimine (a “hydrophilic component”) that has been linked to one or more hydrocarbon groups having between 5 and 30 carbon atoms linearly arranged (“hydrophobic component(s)”, which may be linked to the hyperbranched polyethyleneimine using any of a number of different linkages). In another embodiment, the hyperbranched polyethyleneimine core also comprises one or more additional organic “cap” compounds attached to the h-PEI. The typical add-on weight of the h-PEI derivative is from about 0.1% of the weight of the fabric to about 10% of the weight of the fabric and, preferably, is from about 0.2% of the weight of the fabric to about 5% of the weight of the fabric.

In schematic terms, the h-PEI molecule can be described as having a central core surrounded by a plurality of molecular branches, with each branch projecting outward from the core and having a highly reactive end group. It is to be expected that partial linkage of the branches to themselves often occurs. The molecule typically exhibits a very high charge density per area, meaning that there are a high number of positive charges clustered densely together around the molecular core. This configuration makes the molecule very capable of interacting with a wide range of other molecules, many of which will be described herein. The number of molecules that may be attached to the h-PEI molecule depends on the number average molecular weight (Mₙ) of the h-PEI, which reflects the number of branches available for attachment.

Preferably, the hydrophobic components on the h-PEI (that is, hydrocarbon groups and optional linking compounds) are electrophilic, so that they react with the nucleophilic hyperbranched polyethyleneimine molecule. Any of a number of acceptable linking groups may be used to link the hydrocarbon groups to the hyperbranched polyethyleneimine, or the hydrocarbon groups may link directly to the hyperbranched polyethyleneimine molecule.

To the h-PEI molecule are attached at least one, and preferably more than one, hydrocarbon groups to increase the hydrophobicity of the resulting compound (i.e., the h-PEI derivative). These hydrocarbon groups, together with any linking compounds which may be used to attach them to the h-PEI molecule, are collectively referred to as the “hydrophobic components” of the dye-reactive molecule. These hydrocarbon groups may be linear molecules or may contain branched or aromatic portions, which have an electrophilic group capable of reacting with the nucleophilic h-PEI. Preferably, regardless of the structure of the hydrocarbon, the linear portion of the hydrocarbon group contains between about 5 and about 30 carbon atoms and, more preferably, contains between about 10 and about 24 carbon atoms. Mixtures of various length hydrocarbons may also be used.

Examples of electrophilic hydrocarbons include, without limitation, carboxylic acids, ketene dimers, formates, acetyl halides (such as acetyl chloride), esters, anhydrides, alkyl halides, epoxides, isocyanates, and the like. Preferred examples include stearic acid, hydroxy stearic acid, isostearic acid, and palmitic acid.

In one embodiment, the hydrocarbon groups comprise up to 20% (preferably about 0.1% to 20%) of the weight of the hyperbranched polyethyleneimine derivative and more preferably, from about 2% to about 15% of the weight of the h-PEI derivative. In such embodiment, the treated textile exhibits wicking properties that are desired for textiles used in apparel and other applications.

In a second embodiment, the hydrocarbon groups comprise up between about 20% and about 80% of the weight of the hyperbranched polyethyleneimine derivative, more preferably, from about 30% to about 75% of the weight of the h-PEI derivative. In such embodiment, it has been found that durability may be achieved without the use of a separate cross-linking agent or compound, although one may be incorporated if so desired for certain applications. Textiles treated with derivatives having a greater amount of hydrocarbon groups tend to exhibit finishes that are more water-repellent, which may be useful in some circumstances.

The weight ratio of h-PEI to hydrophobic groups is from about 1000:1 to about 5:1 and, more preferably, is from about 100:1 to about 10:1. In another embodiment, the weight ratio of h-PEI to hydrophobic groups is from about 5:1 to about 1:10 and, more preferably, is from about 2:1 to about 1:5, depending on the Mₙ of the h-PEI. Most preferably, weight ratios of h-PEI to hydrophobic group from about 1:1 to about 1:4 are used.

The present h-PEI derivatives possess the structure shown below:

(R)ₓ−h-PEI−(A)_{y}

where R is a non-hyperbranched hydrocarbon group (for example, such as alkyl, alkenyl, arylalkyl, and arylalkenyl groups, where the number of carbon atoms in the linear portion of the hydrocarbon is between 5 and 30 carbon atoms), where x is a number from 1 to about 10,000 (depending on the Mₙ of the h-PEI), where h-PEI is a hyperbranched polyethyleneimine, where A is a small organic “capping” compound, where y is a number from 0 to 500, and wherein R is present in an approximate amount of between about 0.1% and about 80% by weight of the molecule. It is understood that, in the synthesis of molecules such as those fitting the general structure provided above, the actual product exhibits a polydispersity (a distribution of ratios) and the molar ratio of each molecule will vary somewhat around the target ratio. For the applications that are contemplated herein, hyperbranched polyethyleneimines having a number average molecular weight (Mₙ) of between about 300 and about 2 million are preferred, with Mₙ of between about 1,000 and about 75,000 being more preferred.

Optionally, small organic molecules (generically shown as “A” in the structure above) may be used to “cap” the unreacted branches of the hyperbranched polyethyleneimine. Generally speaking, the capping molecule “A” has from one to four carbon atoms. Any caps that will react with the amine (NH or NH₂) portion of the h-PEI molecule may be used, including, without limitation, epoxides, anhydrides, esters, acids, carbonates, sulfates, formates, isocyanates, and mixtures thereof. Specific examples of such caps include, without limitation, ethylene oxide, propylene oxide, methyl bromide, acetic acid, vinyl sulfonates, trifluoroacetic acid, and succinic anhydride. Mixtures of different “cap” molecules may be used.

Ethylene oxide (EO) or propylene oxide (PO) chains are especially useful as capping compounds in the present treatment to prevent the treated substrate from yellowing when exposed to high manufacturing temperatures (for example, temperatures greater than 350° F.). The addition of such chains is not required to achieve odor control and other benefits of the present treatment, but merely to impart additional benefits. For example, it has been found that the addition of EO or PO chains within the h-PEI derivative improves moisture wicking and produces softness in the treated substrate.

One potentially preferred “R” group has a C₁₇H₃₅ structure, which in the above structure forms a stearic amide. When using this R group and an h-PEI having an Mₙ of 1200, representative molar ratios of h-PEI to R are 1:2, 1:4, 1:6, 1:8, 1:10, and 1:12. Similarly, when the Mₙ of the h-PEI molecule is about 10,000, representative molar ratios of h-PEI to R are 1:25, 1:60, 1:80, and 1:100. Finally, when the Mₙ of the h-PEI molecule is about 75,000, representative molar ratios of h-PEI to R are 1:400, 1:500, and 1:600.

The chemical synthesis of the present treatment molecules is conducted by reacting the h-PEI molecule with a hydrophobic R-containing electrophilic molecule in the presence of nitrogen. It has been found that mechanical agitation of the reagents in a vessel under nitrogen at a temperature of about 150° C. produces the h-PEI derivatives described herein. The time necessary to complete the reaction depends on the amount of reagents that are being reacted and the size of the reaction vessel. The resulting compounds, referred to herein as “h-PEI derivatives”, are typically in the form of an oily liquid or waxy solid.

To prepare a treatment bath for textiles using the h-PEI derivatives described herein, one approach is to heat the h-PEI derivative to its melting point, so that it may be poured into a vessel where it is combined, via high speed and high shear agitation, with hot water. In this instance, the phrase “hot water” refers to water having a temperature equal to or greater than the melting point of the h-PEI derivative. Suitable equipment for achieving high speed and high shear agitation includes propeller-type mixers, Jago®-type agitators, homogenizers, roll mill, ball mill, microfluidization, and the like.

The dispersion that results from the forcible introduction of the h-PEI derivative into water may be assisted and stabilized by addition of a solubilizing agent (e.g., an acid or a surfactant), the amount of which depends on the molecular weight of the h-PEI and the molar ratio of h-PEI to hydrophobic components. Acetic acid is one potentially preferred acid for this purpose (excess acid being evaporated off during subsequent drying of the treated textile substrate). Amounts of greater than 0.1% acid, by weight of solution, may be used successfully. Preferably, the amount of acid will be in the range of about 0.1% to about 50% of the weight of the h-PEI derivative.

Without wishing to be bound by theory, it is hypothesized that the h-PEI derivatives disclosed herein possess a molecular configuration that facilitates odor adsorption. Specifically, the h-PEI derivatives have a hydrophilic core surrounded by a hydrophobic “shell” that is formed by the plurality of hydrocarbon groups attached to the core h-PEI molecule. Such a configuration results in numerous voids within the derivative molecule, in which volatile odor molecules having different polarities may be trapped. Additionally, secondary interactions—such as, for example, Van der Waals forces, hydrogen bonding, and ionic interactions—may also contribute to the odor-trapping ability exhibited by textiles treated with the present derivatives. Additional information about h-PEI may be found in pending applications Ser. Nos. 11/651,711 and 11/651,688, incorporated herein by reference.

Another component of the finish on the fabric is a compound selected from the group consisting of silver particle-containing compounds, silver ion-containing compounds, silver ion-generating compounds, and any combinations thereof a non-electrically conductive silver-ion containing compound. The silver-ion containing compound is selected from the group consisting of silver zirconium phosphate, silver zeolite, silver glass, and any mixtures thereof. The term silver-ion containing compounds encompass compounds which are either ion-exchange resins, zeolites, or, possibly substituted glass compounds (which release the particular metal ion bonded thereto upon the presence of other ionic-species). The preferred silver-ion containing compound for this invention is an antimicrobial silver zirconium phosphate available from Milliken & Company, under the trade name ALPHASAN®. Other potentially preferred silver-containing antimicrobials in this invention is a silver zeolite, such as those available from Sinanen under the trade name ZEOMIC® AJ, or a silver glass, such as those available from Ishizuka Glass under the trade name IONPURE®, may be utilized either in addition to or as a substitute for the preferred species. Generally, such a metal compound is added in an amount of from about 0.01 to about 40% by total weight of the particular treatment composition; more preferably from about 0.05 to about 30%; and most preferably from about 0.1 to about 30%. Preferably this metal compound is present in an amount of from about 0.01 to about 5% owf, preferably from about 0.05 to about 3% owf, more preferably from about 0.1 to about 2% owf, and most preferably about 1.0% owf. More information on the silver based finishes may be found in U.S. Pat. No. 7,132,378, incorporated herein by reference.

Preferably, the treated fabric exhibits a silver-ion release retention level of at least 5%, with an initial amount of available silver ion of at least 1000 ppb, as measured by a phosphate buffer comparison test. The silver-ion release retention level is measured after at least 10 washes, the washes being performed in accordance with the wash procedure as part of a modified AATCC Test Method 130-1981 at least 120° F. In another embodiment, the treated fabric exhibits a log kill rate for Staphylococcus aureus after 24 hour exposure in accordance with MTCC Test Method 100-1993 of at least 1.5. The log kill rate is measured after at least 10 washes, the washes being performed in accordance with the wash procedure as part of a modified MTCC Test Method 130-1981 at least 120° F. In another embodiment of the invention, the treated fabric has a molar ratio of halide ions to silver ions is within the range of from 1:10 to 5:1, and the finish is substantially free from alkali metal ions.

The compound may also be silver particle-containing compounds. One such example is a conductive silver containing nanoparticle. Additional information on silver containing compounds may be found in U.S. Pat. No. 7,291,570, incorporated herein by reference.

The cross-linked agent in the finish provides highly beneficial durability for the coated fabrics. Preferably, this component is a polyurethane-based binding agent, although other types, such as a permanent press type resin or an acrylic type resin, may also be utilized in combination, particularly, with the optional halide ion additive for discoloration reduction. The cross-linking agent utilized therewith may be selected from the group consisting of urea-based types, blocked isocyanates, epoxy-based compounds, melamine-formaldehydes, alkoxyalkylmelamines, and any mixtures thereof. Multifunctional cross-linking agents are particularly preferred for this invention. Such compounds generally exhibit an average of at least three reactive groups per molecule, thereby permitting higher efficiency and density for stronger and more reliable cross-linking capabilities. Specific types of cross-linking agents useful within this invention include (with non-limiting examples of such specific types within parentheses) modified ethylene urea (such as FREEREZ® PFK, from Freedom Textile Chemical, having about 44% solids content), blocked isocyanates (such as REPEARL® MF, from Mitsubishi International Corporation, having about 36% solids content), polyisocyanates (such as BAYHYDUR® 302, from Bayer, having about 99.8% solids content), epoxies (such as EPIREZ® 5003, from Resolution Performance Products, having about 55% solids content), melamine-formaldehyde condensates (such as AEROTEX® M3, from Noveon, having about 80% solids content), methylated melamine-formaldehydes (such as CYMEL® 301, from Cytec Industries, having about 98% solids content), and hexamethoxymethylmelamines (such as CYMEL® 385, having about 80% solids content), and carbodiimides. In one preferred embodiment, the cross-linking agent is a block polyisocyanate cross-linking agent. The EPIREZ types (as listed above), as an example, exhibit a functionality of three for, as noted previously, stronger cross-linking capabilities, and therefore are exceptionally good for these desired characteristics. Alternatively, difunctional cross-linking agents, with high concentrations of reactive groups per unit weight are also possible. For example, a certain weight (grams) of resin containing one gram-equivalent of epoxide (otherwise known as WPE), characterizes the concentration of epoxide reactive groups. The aforementioned EPIREZ 5003 exhibits a WPE of 200, which is, as noted, highly effective. Such resins, epoxy or otherwise, with WPE measurements of 500 or less would thus be suitable for this invention. Most preferred would be those having a WPE less than about 250.

Another component of the finish on the coated fabric is a polyurethane binder. Within the particular topical application procedures, the initial application of the silver-ion compound (preferably, ALPHASAN®) and p-HEI is thus preferably followed by a thin coating of cross-linked polyurethane-based binder resin to provide the desired high temperature wash durability characteristics for the silver-ion based antimicrobial and odor reducing treatment. With such specific cross-linked polyurethane-based binder materials utilized, the antimicrobial characteristics of the treated fabric remained very effective for the fabric even after as many as ten high temperature laundering procedures. While not being bound to any particular theory, it is envisioned that the thin coating of polyurethane-based resin, which is bound to the fabric, is also attached to the cross-linker, which is itself attached to the antimicrobial agent, thereby providing long-term wash durability. This indirectly also helps provide long-term wash durability to the odor adsorption finish.

Also possible, and more effective in most situations as compared to the aforementioned binder resin overcoat, but still an acceptable method of providing a wash-durable antimicrobial, odor adsorbing fabric, is the application of a finish containing the polyurethane as a component rather than as an overcoat (coated from a pad bath mixture followed by nip roll wringing of excess liquor and high temperature drying thereof). The contacting of such a combination is less efficacious from an antimicrobial activity standpoint than the other overcoat, but, again, still provides a wash-durable treatment with acceptable antimicrobial benefits. This mixture of compound/resin may also be applied through spraying, dipping, exhaustion, and the like.

In terms of discoloration, it was noticed that silver-ion topical treatments were at times susceptible to yellowing, browning, graying, and, possibly, blacking after exposure to atmospheric conditions. As silver ions are generally highly reactive with free anions, and most anions that react with silver ions produce color, a manner of curtailing if not outright preventing problematic color generation upon silver ion interactions with free anionic species, particularly within dye bath liquids, was required. Thus, it was theorized that inclusion of an additive that was non-discoloring itself, would not react deleteriously with the cross-linked binder and/or silver-ion compound, and would, apparently, and without being bound to any specific scientific theory, react in such a manner as to provide a colorless salt with silver ions, was highly desired. Halide ions, such as from magnesium chloride, a metal halide, have been found to reduce or eliminate the yellowing of the silver ions. Inorganic chlorides are preferred, especially magnesium chloride and ammonium chloride. A range of ratios from 1:10 (chloride to silver ion) to 5:1 (chloride to silver ion) should be met for proper activity; preferably this range is from 1:2 to about 2.5:1. Again, higher amounts of metal halide in molar ratio to the silver ions may be added to counteract any excess alkali metal ion amounts within the finish composition itself.

Additionally, it was found that the h-PEI derivatives also suffered from discoloration and it was found that potassium citrate reduced or eliminated the yellowing of the h-PEI. Preferably, potassium citrate is added to the fabric is an amount of between about 0.05 and 10% owf, more preferably 0.1 to 1.0% owf.

The fabric substrate may be dyed or colored to provide other aesthetic features for the end user with any type of colorant, such as, for example, poly(oxyalkylenated) colorants, as well as pigments, dyes, tints, and the like. Other additives may also be present on and/or within the target fabric or yarn, including antistatic agents, brightening compounds, nucleating agents, antioxidants, UV stabilizers, fillers, permanent press finishes, softeners, lubricants, curing accelerators, and the like. Particularly desired as optional and supplemental finishes to the inventive fabrics are soil release agents which improve the wettability and washability of the fabric. Preferred soil release agents include those which provide hydrophilicity to the surface of polyester. With such a modified surface, again, the fabric imparts improved comfort to a wearer by wicking moisture. The preferred soil release agents contemplated within this invention may be found in U.S. Pat. Nos. 3,377,249; 3,540,835; 3,563,795; 3,574,620; 3,598,641; 3,620,826; 3,632,420; 3,649,165; 3,650,801; 3,652,212; 3,660,010; 3,676,052; 3,690,942; 3,897,206; 3,981,807; 3,625,754; 4,014,857; 4,073,993; 4,090,844; 4,131,550; 4,164,392; 4,168,954; 4,207,071; 4,290,765; 4,068,035; 4,427,557; and 4,937,277. These patents are accordingly incorporated herein by reference. Additionally, other potential additives and/or finishes may include water repellent fluorocarbons and their derivatives, silicones, waxes, and other similar water-proofing materials.

The preferred procedure to create the antimicrobial and odor adsorbing fabric includes obtaining a textile and forming a bath containing a compound selected from the group consisting of silver particle-containing compounds, silver ion-containing compounds, silver ion-generating compounds, and any combinations thereof, a hyperbranched polyethyleneimine derivative, potassium citrate, inorganic chloride, a polyurethane binder, and a cross-linking agent. The target fabric is then immersed in the pad bath. Subsequently, the treated fabric is then squeezed through a nip roll and dried at a temperature between 160 and 400° F. depending on the nature of the fabric end-use.

### EXAMPLES

The following examples further illustrate the present invention but are not to be construed as limiting the invention as defined in the claims appended hereto. All parts and percents given in these examples are by weight unless otherwise indicated.

Formation of h-PEI

To a round-bottom flask with a mechanical agitator were added 200.0 grams of hyperbranched polyethyleneimine (sold under the name EPOMIN® SP012 by Summit Specialty Chemical, New Jersey) and 94.83 grams of stearic acid (sold by Aldrich, Wisconsin). The hyperbranched polyethyleneimine had a Mₙ of 1200. The mixture was heated under nitrogen, with agitation, at a temperature of about 150° C. for about 3 hours. At the end of the 3 hours, an aliquot was removed and analyzed using FT-IR, which indicated that no acid remained and that the reaction was complete. The resulting product was a waxy solid.

The h-PEI derivative was dispersed into hot water via high speed and high shear agitation. To solubilize the h-PEI derivative, acetic acid was added to the dispersions to achieve a pH level of about 5. The h-PEI derivative comprised about 3.0% by weight of the dispersion. Also added to the dispersion was about 2.0% by weight of a blocked isocyanate cross-linking agent (available from Clariant Corporation under the trade name ARKOPHOB® DAN). The resultant product is designated as h-PEI in the following examples.

### Formation of Treated Fabrics

Two formulas of antimicrobial and odor adsorbing finishes were created:

TABLE 1
Formula 1

| Formula 1 | | wet pick up = 79% | |
|---|---|---|---|
| Chemical | | % owf | % in finish |
| Tap water | | | 90.51% |
| Potassium | | 0.60% | 0.76% |
| citrate | | | |
| Magnesium | | 0.50% | 0.63% |
| chloride | | | |
| ALPHASAN ® | Silver zirconium phosphate | 0.40% | 0.51% |
| RC5000 | avail. from Milliken and Co. | | |
| Dousoft OH ® | Silicone softener avail. | 1.50% | 1.90% |
| | from Dystar | | |
| MRX ® | Block isocyanate cross-linking | 1.00% | 1.27% |
| | agent avail. from Milliken | | |
| | Chemical | | |
| M293 ® | Polyurethane binder avail. | 1.00% | 1.27% |
| | from Milliken Chemical | | |
| h-PEI | Formed as described above | 0.50% | 3.16% |
| | with % active = 20% | | |

TABLE 2
Formula 2

| Formula 2 | | wet pick up = 79% | |
|---|---|---|---|
| Chemical | | % owf | % in finish mix |
| Tap water | | | 90.52% |
| Potassium citrate | | 0.60% | 0.76% |
| Magnesium | | 0.50% | 0.63% |
| chloride | | | |
| ALPHASAN ® | Silver zirconium phosphate | 0.40% | 0.51% |
| RC5000 | avail. from Milliken | | |
| | and Co. | | |
| Dousoft OH ® | Silicone softener avail. | 1.50% | 1.90% |
| | from Dystar | | |
| W-XW ® | Epoxide avail. from | 0.20% | 1.25% |
| | Chemtura | | |
| M293 ® | Polyurethane binder avail. | 1.00% | 1.27% |
| | from Milliken Chemical | | |
| h-PEI | Formed as described above | 0.50% | 3.16% |
| | with % active = 20% | | |

The two finishes were applied by padding on the finish on a 100% polyester circular knit fabric to form Example 1 and Example 2 (with finish 1 and finish 2 on the fabrics, respectively). The fabric was heat set at 360 F for three minutes. The treated fabrics were then subjected to home laundering (HL) and tested after 0, 10, and 25 HL. The home laundering conditions were washing at 75 F for 10 minutes under the small load conditions using Tide and no ballast followed by 10 minutes of normal dryer heat.

### Testing

Antimicrobial testing to test the antimicrobial and silver containing ion included silver counts as both an XRF value and as bioavailable silver. These tests were performed on as received treated fabric and washed treated fabric.

Odor control was measured by a GC headspace analysis technique. In this method a sample of fabric is placed in a sealed vial which is then spiked by an odor cocktail containing isovaleric acid. Isovaleric acid is a short chain fatty acid and a major component of foot and auxiliary odor. After equilibrating for approximately one hour at 37 C, a fiber is inserted into the GC and the amount of molecules unbound to the fabric is measured. Durability is defined as a 70% reduction of isovaleric acid through 25 HL on PET/nylon and a 50% reduction through 25 HL on a PET knit fabric.

### Results

TABLE 3
XRF test results

| | # of HL | # of Ag | # of ZR | % Ag after | % Zr after |
|---|---|---|---|---|---|
| Sample | washes | counts | counts | washes | washes |
| Example 1 | 0 | 70 | 390 | | |
| Example 1 | 10 | 57 | 276 | 81% | 71% |
| Example 2 | 0 | 100 | 498 | | |
| Example 2 | 10 | 53 | 269 | 53% | 54% |

TABLE 4
Bioactive silver results

| Sample | # of HL washes | ppm Ag |
|---|---|---|
| Example 1 | 25 | 0.055 |
| Example 2 | 25 | 0.088 |

TABLE 5
Odor absorption as determined by GC headspace analysis

| | # of HL | | % reduction |
|---|---|---|---|
| Sample | washes | Isovaleric area | vs control |
| Control (fabric with no finish) | 25 | 217 | |
| Example 1 | 0 | 1 | 100% |
| Example 1 | 10 | 46 | 79% |
| Example 1 | 25 | 67 | 69% |
| Example 2 | 0 | 1 | 100% |
| Example 2 | 10 | 62 | 71% |
| Example 2 | 25 | 91 | 58% |

As one can see from the above data, the combination of a fabric with a finish containing a non-electrically conductive silver-ion containing compound, a hyperbranched polyethyleneimine derivative, potassium citrate, magnesium chloride, a polyurethane binder, and a cross-linking agent as described above, produces an antimicrobial and odor adsorbing fabric.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. An antimicrobial and odor adsorbing fabric substrate having a surface, at least a portion of which is coated with a finish comprising:
a compound selected from the group consisting of silver particle-containing compounds, silver ion-containing compounds, silver ion-generating compounds, and any combinations thereof;
a hyperbranched polyethyleneimine derivative of the formula:
(R)ₓ−h-PEI−(A)_{y}
where R is a non-hyperbranched hydrocarbon group, the hydrocarbon group has at least one linear portion, the linear portion having between 5 and 30 carbon atoms; where x is a number from 1 to 10,000; where h-PEI is a hyperbranched polyethyleneimine; where A is an organic compound having from 1 to 4 carbon atoms; where y is a number from 0 to 500;
potassium citrate;
inorganic chloride;
a polyurethane binder; and,
a cross-linking agent.

2. The antimicrobial and odor adsorbing fabric substrate of claim 1, wherein R is present in an amount of between about 0.1% and about 20% by weight of said hyperbranched polyethyleneimine derivative.

3. The antimicrobial and odor adsorbing fabric substrate of claim 1, wherein R is present in an amount of between about 20% and about 80% by weight of said hyperbranched polyethyleneimine derivative.

4. The antimicrobial and odor adsorbing fabric substrate of claim 1, wherein the finish is applied to the fabric substrate at an add-on level of between about 0.2% to about 5%, based on the weight of the substrate.

5. The antimicrobial and odor adsorbing fabric substrate of claim 1, wherein the h-PEI has a number average molecular weight (Mₙ) in range of about 1,000 to about 75,000.

6. The antimicrobial and odor adsorbing fabric substrate of claim 1, wherein the linear portion of said R group has between about 10 and about 24 carbon atoms.

7. The antimicrobial and odor adsorbing fabric substrate of claim 1, wherein the h-PEI and the R group are present, in a weight ratio, of from about 100:1 to about 10:1.

8. The antimicrobial and odor adsorbing fabric substrate of claim 1, wherein the A group is at least one compound selected from the group consisting of ethylene oxide, propylene oxide, butylene oxide, methyl, acetate, vinyl sulfonate, trifluoroacetate, and trialkyl silyl.

9. The antimicrobial and odor adsorbing fabric substrate of claim 1, wherein the R group has a C₁₇H₃₅ structure, resulting in a stearic amide linkage between the R group and the h-PEI.

10. The antimicrobial and odor adsorbing fabric substrate of claim 1, wherein the cross-linking agent is an isocyanate cross-linking agent.

11. The antimicrobial and odor adsorbing fabric substrate of claim 1, wherein the silver-ion release retention level is at least 80%.

12. The antimicrobial and odor adsorbing fabric substrate of claim 1, wherein the coated fabric substrate exhibits a log kill rate for Staphylococcus aureus after 24 hour exposure in accordance with AATCC Test Method 100-1993 of at least 1.5, wherein said log kill rate is measured after at least 10 washes, said washes being performed in accordance with the wash procedure as part of a modified AATCC Test Method 130-1981 at at least 120° F.

13. The antimicrobial and odor adsorbing fabric substrate of claim 1, wherein the molar ratio of halide ions to silver ions is within the range of from 1:10 to 5:1, and wherein the finish is substantially free from alkali metal ions.

14. The antimicrobial and odor adsorbing fabric substrate of claim 1, wherein the finish is wash durable.

15. The antimicrobial and odor adsorbing fabric substrate of claim 1, wherein the inorganic chloride is magnesium chloride.

16. The antimicrobial and odor adsorbing fabric substrate of claim 1, wherein the inorganic chloride is ammonium chloride.

17. The antimicrobial and odor adsorbing fabric substrate of claim 1, wherein the compound is silver zirconium phosphate.

18. A process of forming an antimicrobial and odor adsorbing fabric substrate comprising:
(a) providing a fabric substrate;
(b) providing a dispersion comprising a compound selected from the group consisting of silver particle-containing compounds, silver ion-containing compounds, silver ion-generating compounds, and any combinations thereof, potassium citrate, inorganic chloride, a polyurethane binder, a cross-linking agent, and a hyperbranched polyethyleneimine derivative of the formula:
(R)ₓ−h-PEI−(A)_{y}
where R is a non-hyperbranched hydrocarbon group, the hydrocarbon group has at least one linear portion, the linear portion having between 5 and 30 carbon atoms, where x is a number from 1 to 10,000, where h-PEI is a hyperbranched polyethyleneimine, where A is an organic compound having from 1 to 4 carbon atoms, where y is a number from 0 to 500, and wherein the hyperbranched polyethylene derivative being produced by:
(i) providing said hyperbranched polyethyleneimine derivative, a solubilizing agent, and water, said water having a temperature at least equal to the melting point of said hyperbranched polyethyleneimine derivative; and
(ii) subjecting said hyperbranched polyethyleneimine derivative, said solubilizing agent, and said water to high speed and high shear agitation;
(c) applying said dispersion to said textile substrate; and
(d) drying said textile substrate.
